(19) United States
(12) Patent Application Publication
(10) Pub. No.: US 2003/0149438 A1
(43) Pub. Date: Aug. 7, 2003

(54) Insertion instrument

(75) Inventors: David Nichols, Trumbull, CT (US); John R. Pepper, Cheshire, CT (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Howmedica Osteonics Corp., Allendale, NJ (US)

(21) Appl. No.: 10/321,683

(22) Filed: Dec. 17, 2002

Related U.S. Application Data
(63) Continuation of application No. PCT/US02/13472, filed on Apr. 30, 2002.
(60) Provisional application No. 60/351,246, filed on Jan. 23, 2002.
(60) Provisional application No. 60/351,247, filed on Jan. 23, 2002.
(60) Provisional application No. 60/286,913, filed on Apr. 30, 2001.

(51) Int. Cl. A61B 17/88; A61B 17/70

(52) U.S. Cl. 606/99; 606/61; 623/17.16

(57) ABSTRACT

There is provided an insertion instrument that has a first, partially hollow elongated member and a second elongated member that can be positioned relative to the first member. The two members can be locked together and dissociated from each other via a turnbuckle locking mechanism. Each member has a tine protruding at its distal end. When the two members are locked together, the two tines form a fork-like front end for holding an implant during insertion. One tine may be retracted to release the implant in a desired position. There is also provided a method for holding, accurately positioning and inserting the implant into an intervertebral space between adjacent vertebrae. There is further provided a mechanism and method for releasing the implant between adjacent vertebrae, and easily and safely removing the insertion instrument.

### RELATED APPLICATIONS

This application is a continuation of International Application No. PCT/US01/13472, which was filed on Apr. 30, 2002 and claims priority to the following U.S. provisional applications, all of which are now hereby incorporated by reference in their entireties: U.S. Application No. 60/351,246 filed on Jan. 24, 2002, U.S. Application No. 60/351,247 filed on Jan. 24, 2002 and U.S. Application No. 60/286,913 filed on Apr. 30, 2001.

### BACKGROUND OF THE INVENTION

The present invention generally relates to a device for use as a surgical instrument. In a particular embodiment, the present invention relates to a device that is adapted to hold a surgical implant, such as a bone graft, and is used to seat the surgical implant or bone graft into a designated vertebral disc space.

Many orthopedic procedures involve the insertion of either natural or prosthetic implants into or adjacent to bone or associated tissues. For example, ligament repair, joint repair or replacement, non-union fractures, facial reconstruction, spinal stabilization and spinal fusion are all such procedures. Typically in such procedures, an insert, dowel or screw is inserted into a prepared bore formed in the bone or tissues.

Surgical instruments for spinal fusion implant insertion are known. Some patents provide for an apparatus that has an outer and an inner sleeve arrangement. The outer sleeve is positioned over the spinal distractor and has teeth at one end that are driven directly into the posterior surface of the adjacent vertebrae. The inner sleeve is positioned within the outer sleeve and serves to guide instruments, such as a drill, used to form the implant receiving bore. Other arrangements include the use of guide rods that are placed in pilot holes formed in the vertebral bodies. The guide rods guide a bore forming hollow drill into the intevertebral space.

When installing these specialized implants, an insertion tool is used to position the implant in the desired intervertebral location. Once the implant is in position, the insertion tool is removed.

Although some current instrumentation and methods associated therewith for the placement of spinal fusion implants have been generally effective for their intended purposes, there exist certain limitations with the design of such instrumentation that detracts from their usefulness. For example, an implant can slip or move during insertion, which may result in injury to tissues and structures in the spine or neck. Accordingly, a. need exists for an insertion tool and method that facilitate safe and effective implant insertion and placement.

### SUMMARY OF THE INVENTION

It is an object of the present invention to provide an effective and safe instrument for inserting an implant.

It is another object of the present invention to provide an insertion instrument and associated method that is used for placement of a bone graft or allograft implant between adjacent vertebrae.

These and other objects and advantages of the present invention are achieved by an instrument for insertion of an implant, which instrument preferably has two components removably connected together, with each component having a tine located at its distal end. The two components, when assembled, provide a fork-like front end with two prongs or tines. Each of the two components removably connects to the other by a two-piece turnbuckle locking member that has a separate piece located on each component.

The fork-like front end can be withdrawn one tine at a time, so the implant stays in place while the instrument is removed. The front end can hold a surgical implant on three sides, and keep the implant from rotating and from slipping laterally or vertically when positioning the implant into a designated vertebral disc space. In a preferred embodiment, the first tine moves relative to the second or fixed tine. One tine is released by turning the handle of the instrument preferably about ninety degrees in either a clockwise or counter-clockwise direction, thereby releasing the turnbuckle locking mechanism that serves to secure together and dissociate the two parts of the instrument. The second tine is released only by removal of the instrument.

### BRIEF DESCRIPTION OF THE DRAWINGS

FIG. 1 is a perspective view of the insertion instrument of the present invention;

FIG. 2 is an exploded plan view of the two components of the insertion instrument of FIG. 1;

FIG. 3 is a first plan view of the insertion instrument of FIG. 1, with the instrument shown in the locked or insertion position;

FIG. 4 is a second plan view of the insertion instrument of FIG. 1, with the instrument shown in the unlocked or retraction position, and with the handle rotated ninety degrees;

FIG. 5 is a perspective view of an alternative embodiment of the insertion instrument of the present invention;

FIG. 6 is a perspective view of one embodiment of an implant;

FIG. 7 is a view of the tip of the insertion instrument of FIG. 1 with an implant positioned between the two tines; and

FIG. 8 is an enlarged side view of the insertion instrument of FIG. 7;

FIG. 9 is a partial perspective view of the insertion instrument of FIG. 1 showing tines gripping an implant.

### DETAILED DESCRIPTION

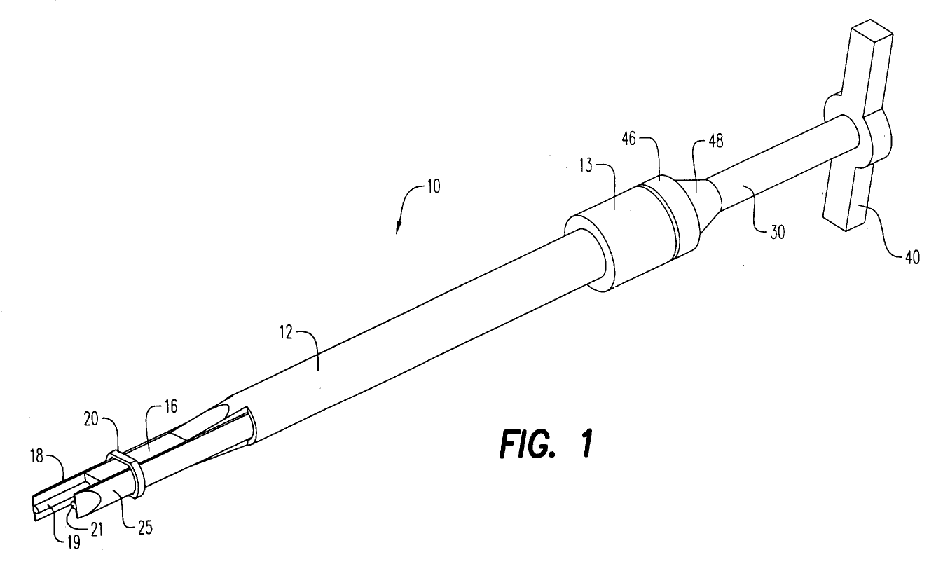

Referring to the drawings and, in particular, FIG. 1, there is shown an implant insertion instrument generally represented by reference numeral 10.

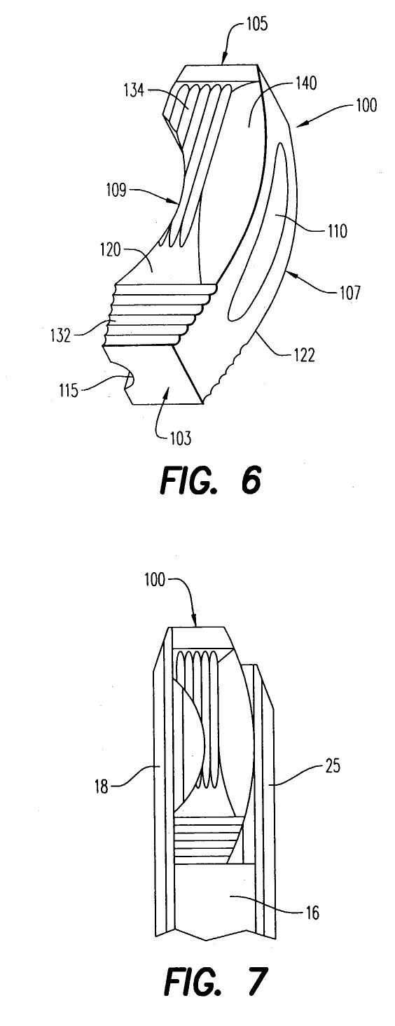

While the insertion instrument and associated method may be employed in a variety of instances, by way of example a preferred embodiment of the insertion instrument 10 is used with a bone fusion implant or allograft to insert an implant such as the one shown in FIG. 6 between vertebrae or bone sections to be fused. The instrument and associated method could be applied in various surgical situations, including those requiring bone fusion or repair. In a preferred embodiment, insertion instrument 10 is loaded with the implant to place the implant in a distracted intervertebral space between adjacent vertebrae.

In the discussion that follows, the term “proximal,” as is traditional, will refer to the portion of the structure that is closer to the operator, while the term “distal” will refer to the portion that is further from the operator.

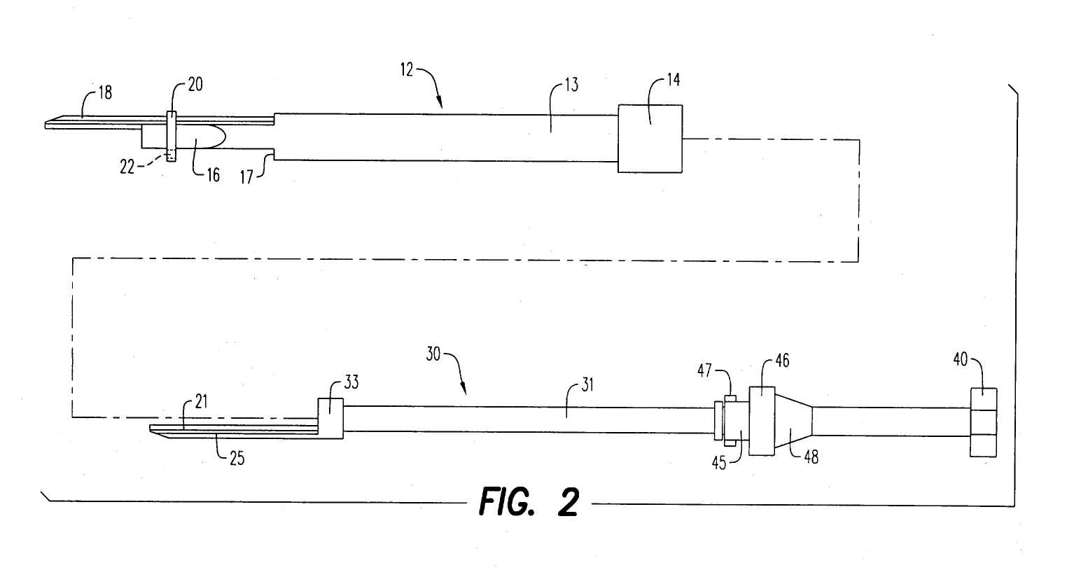

Referring to FIGS. 1 and 2, insertion instrument 10 is a two-piece instrument. Insertion instrument 10 has a partially hollow, elongated member 12 and an elongated member 30 that is at least partially receivable in the elongated member 12.

As shown in FIG. 2, hollow elongated member 12 has a hollow cylindrical section 13 with an enlarged collar 14 at its proximal end, a protrusion 16 towards the distal end, and a first tine 18 adjacent the protrusion 16. Hollow cylindrical member 13 is smaller in diameter than collar 14. Protrusion 16 preferably is generally rectangular in shape.

At the distal end of hollow elongated member 12, there is a first tine 18 that extends across and distally past the length of protrusion 16. On the. other side of protrusion 16, there is a hole 17. The first tine 18 resembles a fork-like tine element, and is positioned virtually or exactly perpendicular to the flat distal side of protrusion 16. Preferably, band 20 encircles first tine 18 and protrusion 16. Band 20 is designed for stabilization of first tine 18. As shown in FIG. 2, there is an open space 22 (shown in phantom) present between the interior surface of band 20 and the outside surface of protrusion 16 that is furthest in distance from first tine 18.

As shown in FIG. 1, first tine 18 preferably has a raised first implant engaging surface or structure 19. Structure 19 is preferably positioned in the center of the inside surface of first tine 18, and extends longitudinally along the length of the first tine in the axial direction. The inside surface of first tine 18 is the surface that comes into contact with the implant. First implant engaging structure 19 is configured to engage a corresponding longitudinal recess or groove in the surface of the implant. First implant engaging structure 19 grips the implant and provides for improved movement and stabilization of the implant during implant insertion.

As shown in FIG. 2, elongated member 30 has a body 31, a handle 40 connected at the proximal end of the body, and a second tine 25 connected at the distal end of the body. Handle 40 has a turnbuckle locking member 45. The most proximal portion of turnbuckle member 45 has a larger diameter than body 31 and preferably forms a stop member 46. Stop member 46 includes a tapered portion 48. Elongated member 30 is preferably generally cylindrical in shape, and has an overall smaller diameter than hollow elongated member 12. Handle 40 rotates relative to body 31.

Elongated member 30 also has a cylindrical front member 33 that is larger in diameter than the remaining portion of body 31 of elongated member 30. As shown in FIG. 2, second tine 25 is located on cylindrical front member 33, preferably at the most distal end of elongated member 30. Second tine 25, which is similar in configuration to first tine 18, also resembles a fork-like tine element. As shown in FIGS. 1 and 2, handle 40 preferably is T-shaped.

As shown in FIGS. 1 and 2, second tine 25 has an inside surface with a second raised implant engaging structure 21 that is positioned in the center of the inside surface. Second implant engaging structure 21 extends longitudinally across the length of second tine 25 in the axial direction.

Elongated member 30 is designed to be removably inserted into hollow elongated member 12. In a preferred embodiment shown in FIG. 2, turnbuckle locking member 45 of elongated member 30 has one or more locking abutments 47. Abutments 47 are designed to latch onto a corresponding area in collar 14.

Referring to FIG. 2, the assembly of elongated members 12 and 30 is as follows. Elongated member 30 can be inserted into hollow elongated member 12 through collar 14. As elongated member 30 is inserted into hollow elongated member 12, second tine 25 passes through hole 17 and then through open space 22 in band 20. Band 20 stabilizes second tine 25 and helps prevent unwanted outward movement. Elongated member 30 is inserted into hollow elongated member 12 until the distal end of stop member 46 contacts collar 14. Once elongated member 30 is fully inserted into hollow elongated member 12, insertion instrument 10 may be locked into operating position by rotating handle 40 preferably approximately ninety degrees in either the clockwise or counter-clockwise direction.

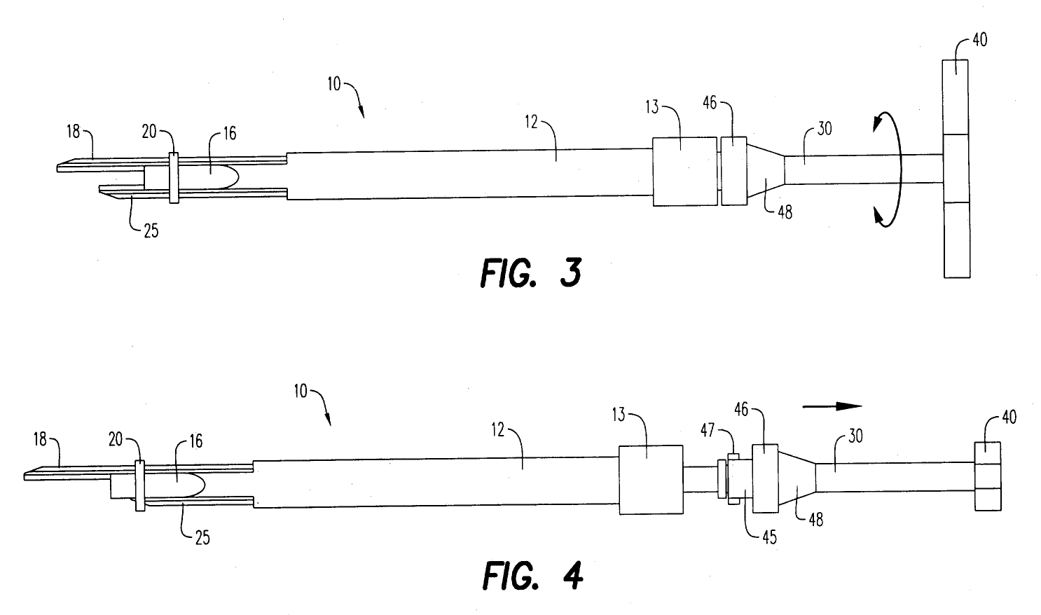

FIG. 3 shows insertion instrument 10 in the locked position, while FIG. 4 shows the insertion instrument in an unlocked, partially separated position. As shown in FIGS. 3 and 4, elongated member 30 may be disengaged from hollow elongated member 12 by turning handle 40 preferably approximately ninety degrees in either the clockwise or counterclockwise direction. By allowing the instrument to be locked or unlocked by turning handle 40 in either the clockwise or counter-clockwise direction, right hand dominant and left hand dominant users of the instrument will find it equally easy to use.

In an alternative embodiment, insertion instrument 10 incorporates a ball detent mechanism to secure elongated member 12 to elongated member 30 and to disengage elongated member 12 from elongated member 30.

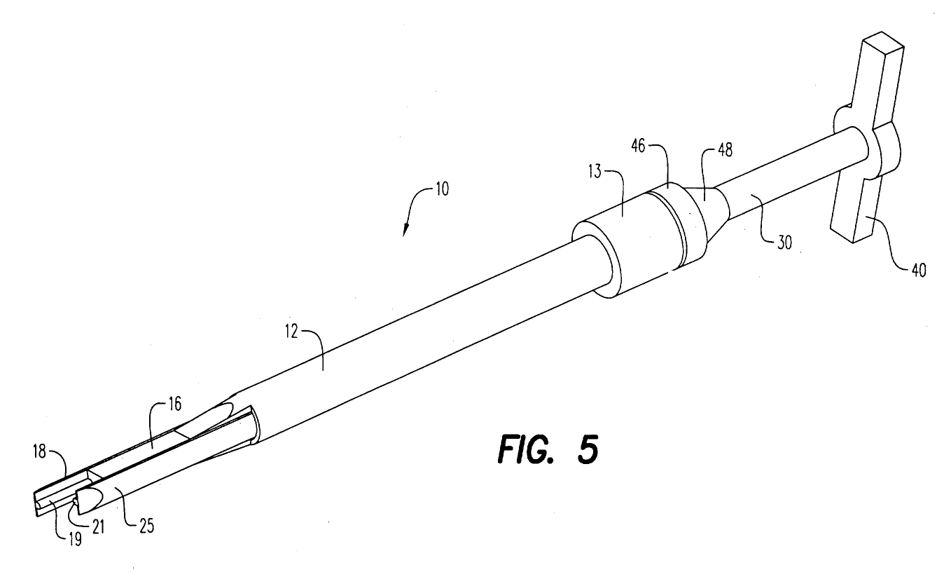

FIG. 5 is an alternative embodiment of insertion instrument 10 that lacks band 20. In this alternative embodiment shown in FIG. 5, first tine 18, second tine 25 and protrusion 16 are not encircled by a band.

The surgical implants used with the present invention are typically used in lumbar interbody fusion and other bone augmentation procedures. The implants used with the present invention may be made from any suitable material, but preferably bone or allograft.

FIG. 6 shows an implant 100 that can be used with insertion instrument 10. Referring to FIG. 6, implant 100 has an anterior side 103, a posterior side 105, a lateral side 107 and a medial side 109. Implant 100 has a groove 110 in the lateral side 107 and groove 115 in the medial side 109 for engaging first implant engaging structure 19 and second implant engaging structure 21 of insertion instrument 10. Groove 110 can be of various configuration, but is preferably of semi-circular cross-section as shown in FIG. 6. The semi-circular section allows for instruments having various diameter shafts to engage and hold the implant 100 after insertion. Implant 100 also has a top or superior surface 120 and an opposite or bottom or inferior surface 122.

As shown in FIG. 6, implant 100 has in the top and bottom surfaces 120, 122, one or more series of grooves. In the preferred embodiment shown in FIG. 6, there is a first or posterior series of grooves 132, and a second or anterior series of grooves 134 substantially perpendicular to the first series. Implant 100 in this embodiment has a top and bottom grooved pattern that is primarily designed to prevent retropulsion of the implant from the intervertebral space. Also, top surface 120 has a planar angled surface 140 as shown.

Implants 100 are of various sizes designed to accommodate the ordinary and usual intervertebral space of patients of different dimensions. The implants 100 are characterized by having a tapered or curved lateral end, which is advantageous to the contour of the body cavity of the intervertebral space. The coronal fit design is a characteristic of implant 100.

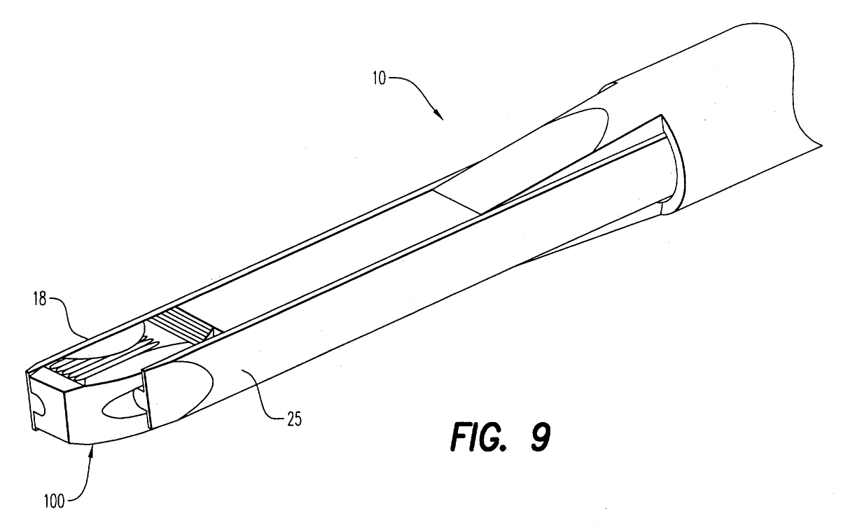

In a preferred embodiment shown in FIGS. 1 and 3, when insertion instrument 10 is in a locked position, second tine 25 preferably is shorter in length distally than first tine 18. In this locked or operating position, first tine 18 and second tine 25 are located parallel to each other with their inside surfaces facing each other, so that their implant engaging structures 19, 21, respectively, engage the implant therebetween. As shown in FIGS. 7 to 9, once in the locked position, an implant or allograft is positioned between the two tines 18, 25 so that the tines hold the bone along the length of the graft. First implant engaging structure 19 and second implant engaging structure 21 are configured to engage corresponding longitudinal recesses or grooves 110 and 115 on the surface of the implant to better hold the implant and provide for improved movement stabilization of the implant during implant insertion. Preferably, distal ends of tines 18, 25 are chamfered or contoured to facilitate insertion into a narrow space and to allow for improved safety during such insertion.

The use of the insertion instrument will now be discussed in connection with a procedure for fusion of vertebral bodies. Prior to introduction of the implant, the intervertebral space has been previously prepared and the vertebrae distracted through the use of distractors and other instruments, as is known in the art. Just prior to insertion of the implant into the intervertebral space, the implant is loaded onto insertion instrument 10 50 that the two tines 18, 25 are juxtaposed on opposing sides of the implant in a horizontal plane. Tines 18, 25, implant engaging structures 19, 21 and the front end of protrusion 16 together control lateral vertical and backward motion and sliding as the implant is inserted into the intervertebral space. The implant is secured laterally by the two tines and proximally by protrusion 16. Undesired vertical motion or sliding is restricted by first implant engaging structure 19 and second implant engaging structure 21. Once the implant is introduced and positioned in the intervertebral space, handle 40 is rotated approximately ninety degrees so that turnbuckle locking member 45 is released and insertion instrument 10 is brought to an unlocked position. By retracting handle 40 in the proximal direction, elongated member 30 is released from hollow elongated member 12 and can be withdrawn away from the surgical area.

After the implant has been positioned in the intervertebral space between the desired adjacent vertebrae, and once elongated member 30 has been retracted, only first tine 18 remains in contact with the implant. For this reason, hollow elongated member 12 can easily be moved away from the implant and can be retracted with virtually no disruption to the positioning of the implant. If required, additional instruments may subsequently be utilized to adjust the positioning of the implant or to further push the implant in the intervertebral space.

As shown in FIGS. 7 to 9, insertion instrument 10 holds an implant or allograft on two sides to hold the bone along the length of the graft. The anterior to posterior holding allows enough force transmitted to reposition insertion instrument 10 intra-operatively. The two thin tines 18, 25 allow enough room to get insertion instrument 10 in past the root and dura without excess distraction.

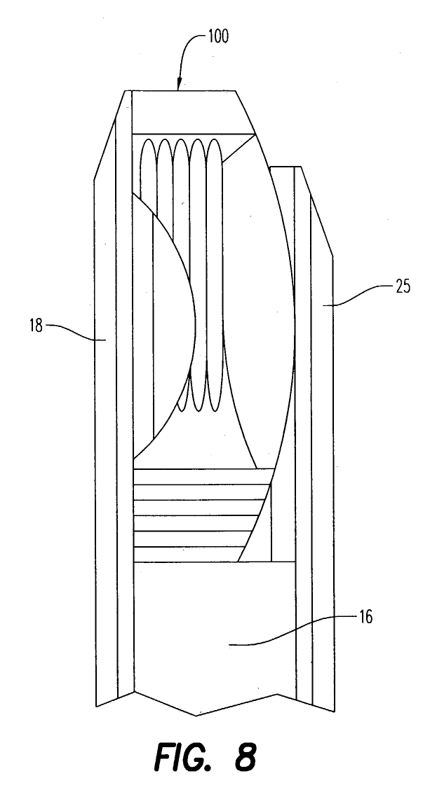

Referring to FIGS. 1 to 5, and 7 to 9, insertion instrument 10 is durable enough to be hammered in place. It is easy to hold. As set forth above, insertion instrument has a method of holding the implant or allograft. The release of tines 18, 25 will allow the instrument to be withdrawn leaving the implant or allograft in place. Tines 18, 25 can be of different lengths to facilitate closer fit to the annulus fibrous, which is curved where insertion instrument 10 will contact it. It is easy to assemble and clean which are important features of hospital equipment. The beveled anterior edge can be seen in FIG. 8.

Insertion instrument 10 is preferably made of biocompatible materials having sufficient strength to withstand the forces encountered during insertion and use. More preferably, insertion instrument 10 may be made of stainless steel, titanium, or aluminum. Since insertion instrument 10 is a two-piece design, it facilitates cleaning and sterilization of the instrument.

The present invention having been thus described with particular reference to the preferred forms thereof, it will be obvious that various changes and modifications may be made therein without departing from the spirit and scope of the present invention as defined herein.

1. A surgical instrument for inserting an implant between a pair of bone structures comprising:
an elongated body having a proximal end and a distal end, said distal end shaped to support the implant at least partially between a first and second extended members of the elongated body, the first and second extended members capable of moving with respect to each other in a generally longitudinal direction to release the implant.

2. The surgical instrument as in claim 1 wherein said elongated body includes a first body structure and a second body structure that is releaseably coupled to said first body structure, and wherein said first extended member is part of said first body structure and said second extended member is part of said second body structure.

3. The surgical instrument as in claim 2 wherein said implant is released by uncoupling said first extended member from said second extended member.

4. A surgical instrument as in claim 1 wherein said first extended member has an engagement surface shaped to mate with a corresponding surface of the implant.

5. A surgical instrument as in claim 4 wherein said engagement surface is shaped to have a linear groove or recess.

6. A surgical instrument as in claim 4 wherein said second extended member also has an engagement surface shaped to mate with another corresponding surface of the implant.

7. An implant insertion apparatus for inserting a bone implant between a pair of adjacent vertebrae comprising:
a first member having a hollow cylindrical section and having a first tine, said first member having a locking mechanism opposite said first tine; and
a second member having a cylindrical configuration that is adapted to be received in the hollow cylindrical section, said second member having a locking abutment to engage said at least one locking mechanism of said first member, said second member having a second tine,
wherein said second member is positioned through the hollow cylindrical section of said first member to connect together said first and second members, wherein said first and second tines of said first and second members, respectively, are parallel to each other when said first and second members are connected together to form a channel for receipt of the bone implant to permit insertion and positioning of the bone implant between the pair of vertebrae.

8. The implant insertion apparatus of claim 7, wherein said first member is removably connected to said second member.

9. The implant insertion apparatus of claim 7, wherein said first member has a protrusion that is located parallel to said first tine.

10. The implant insertion apparatus of claim 9, wherein said protrusion has a distal portion that is rectangular in shape and is positioned in said channel.

11. The implant insertion apparatus of claim 7, wherein said first tine of said first member extends longitudinally past said second tine of said second member when said first and second members are secured together.

12. The implant insertion apparatus of claim 7, further comprising a band for encircling said first and second tines and said protrusion.

13. The implant insertion instrument of claim 12, wherein said first and second tines are positioned parallel to each other and extend in a general longitudinal direction when said first and second members are secured together.

14. The implant insertion apparatus of claim 13, wherein said second tine of said second member protrudes distally in a longitudinal direction through an opening in said first member located adjacent to said protrusion, and wherein said second tine extends distally through said band.

15. The implant insertion apparatus of claim 7, wherein said first tine has a first interior surface for contacting the bone implant.

16. The implant insertion apparatus of claim 15, wherein said second tine has a second interior surface for contacting the bone implant.

17. The implant insertion apparatus of claim 16, wherein said first and second interior surfaces are generally parallel to each other to define said channel.

18. The implant insertion apparatus of claim 15, wherein said first interior surface has a raised first implant engaging structure extending longitudinally on said first interior surface.

19. The implant insertion apparatus of claim 18, wherein said second interior surface has a raised second implant engaging structure extending longitudinally on said second interior surface.

20. The implant insertion apparatus of claim 16, wherein said first and second interior surfaces are substantially planar.

21. The implant insertion apparatus of claim 7, wherein said first and second members have a distal end that is chamfered or contoured.

22. The implant insertion apparatus of claim 7, wherein said second member moves longitudinally and rotationally relative to said first member.

23. The implant insertion apparatus of claim 7, wherein said second member is longitudinally offset relative to said first member.

24. The implant insertion apparatus of claim 7, wherein said second member has a handle.

25. The implant insertion apparatus of claim 7, wherein said second member has a stop arrangement for stopping longitudinal advancement in a distal direction of said second member when inserted into said first member.

26. The implant insertion apparatus of claim 7, wherein said locking mechanism and said locking abutment form a lock.

27. A method for inserting a bone implant between a pair of adjacent vertebrae using the implant insertion apparatus of claim 1, comprising the steps of:
placing the bone implant between said first and second extended members of the implant insertion apparatus;
positioning the bone implant into an intervertebral space by moving the implant insertion apparatus and the bone implant in the intervertebral space;
moving said first extended member with respect to said second extended member in a longitudinal direction to release the implant;
removing said second extended member from the implant.

28. The method of claim 27, wherein said first extended member has a first raised implant engaging structure extending longitudinally on the interior surface of said first extended member, and wherein the method further comprises positioning said first extending member so that said first raised implant engaging structure contacts a first longitudinal recess on a surface of the bone implant.

29. The method of claim 28, wherein said second extended member has a second raised implant engaging structure extending longitudinally on the interior surface of said second extended member, and wherein the method further comprises positioning said second extended member so that said second raised implant engaging structure contacts a second longitudinal recess on a surface of the bone implant.

30. An implant for insertion between adjacent bone structures comprising:
an implant structure having a medial side and a lateral side opposite said medial side, said medial side being shaped to have a concave surface defining a recess, said implant structure shaped to have a groove in at least a portion of either said medial side or said lateral side for engaging a first corresponding structure in an implant insertion device.

31. An implant as in claim 30 wherein said implant structure is shaped to have a groove in at least a portion of the other of said medial side or said lateral for engaging a second corresponding structure in said implant insertion device.

32. An implant as in claim 31 wherein said groove is shaped so that said first corresponding structure is capable of sliding disengaging from said either of said medial side or lateral side.

33. An implant as in claim 30 wherein said medial side is shaped to have a groove that extends through said recess.

34. An implant as in claim 30 wherein said medial side is shaped to have a groove on two opposite sides of said recess.
